(19) United States
(12) Patent Application Publication
(10) Pub. No.: US 2021/0325374 A1
(43) Pub. Date: Oct. 21, 2021

(54) UNIVERSAL MARKER FOR WATER QUALITY ASSESSMENT

(71) Applicant: X2O Corp., New York, NY (US)

(72) Inventor: Alibek Tazhibayev, Somerville, MA (US)

(21) Appl. No.: 17/264,981

(22) PCT Filed: Aug. 1, 2019
(86) PCT No.: PCT/US2019/044634

§ 371 Date: Feb. 1, 2021

Related U.S. Application Data
(60) Provisional application No. 62/714,349, filed on Aug. 3, 2018.

(51) Int. Cl. G01N 33/52; G01N 31/22

(52) U.S. Cl. CPC G01N 33/52; G01N 31/22

(57) ABSTRACT

An apparatus for detecting, in a liquid such as water, concentrations of chemicals that exceed the maximum allowable concentrations, includes a substrate, and a plurality of test strips disposed on the substrate, each test strip having an indicator that reacts to a concentration of a chemical that exceeds the maximum allowable concentration for that chemical. The substrate includes a dissolvable coating covering the test strips to isolate the test strips from air prior to immersion of the substrate in the liquid, and to dissolve when immersed in the liquid to expose the indicator of each test strip to the liquid.

### PRIORITY

This application claims priority from U.S. Provisional Application No. 62/714,349 filed Aug. 3, 2018, entitled “Universal Marker For Water Quality Assessment,” assigned attorney docket number 130658-00101 (formerly 4542/1001), and naming Alibek Tazhibayev as inventor, the disclosure of which is incorporated herein, in its entirety, by reference.

### TECHNICAL FIELD

The present disclosure relates to water testing, and more particularly to devices and method for high speed water quality analysis using chemical indicators.

### BACKGROUND ART

It is known that liquid, such as water, may be contaminated with one or more chemicals. Some chemicals are not harmful to animals that consume such liquids, but some chemicals may be harmful, particularly when concentrated, for example in drinking water. To that end, various organizations, such as the World Health Organization (“WHO”) and the United States Environmental Protection Agency (“EPA”) establish, for each harmful chemical, guidelines and limits for the consumption of such harmful chemicals. For example, the WHO and/or EPA may establish, for each such chemical, a “maximum allowable concentration” (“MAC”) of that chemical in drinking water.

It is known that there are already a variety of portable test systems that allow for water analysis, both in laboratory and field settings, to assess whether the concentration of a chemical in water exceeds the maximum allowable concentration for that chemical. Often these systems are equipped with necessary reagents, indicators and special equipment, such as portable spectrophotometers and photo colorimeters. However, these systems are limited in their usefulness due to their high cost, and the need for a specialized operator. Additionally, such systems do not allow for the simultaneous determination of several elements, require supplemental reagents, and are inaccessible and have minimal availability because of their large size.

Other prior art systems, such as special test kits, are arranged as sets of special reagents and colorimetric lines for rapid analysis of water, using the visual colorimetric method from various sources. However, these methods suffer from a number of disadvantages. For example, they do not allow for simultaneous determination of several elements, they need additional reagents, and there are usage and field limitations brought on by the physical fragility of the glass reaction tubes required.

Furthermore, test strips may be used for the analysis of water and each element. However, like the special test kits above, test strips are not able to determine several elements simultaneously. Therefore, for definitions of up to 10 or 20 elements, the user is required to drop a corresponding number of test strips in the water sample under study, using each time a new sample tube, to guarantee reliable analysis.

### SUMMARY OF THE EMBODIMENTS

In accordance with one embodiment of the present invention, a device for detecting, in a liquid (e.g., water), concentrations of chemicals that exceed a maximum allowable concentration includes a first substrate, a plurality of indicator carriers and at least one atmospheric isolator. Each of the indicator carriers may react to a concentration of a chemical that exceeds the maximum allowable concentration for that chemical. The atmospheric isolators cover each of the plurality of indicator carriers and isolate the indicator carriers from ambient air prior to immersion of the substrate in the liquid.

In some embodiments, the atmospheric isolator(s) includes a plurality of capsules, and one of the indicator carriers is located in each one of the capsules. Each of the capsules may be sealed to the substrate on a first side of the first substrate. Additionally or alternatively, the capsules may be formed in a second substrate, and the second substrate may be sealed to the first substrate on a first side of the first substrate. The capsules may each have a capsule interior and an indicator carrier may be located in each of the capsule interiors. For example, the indicator carriers may be located on an inner wall of its respective capsule.

The first substrate may be transparent to allow for visual inspection of the indicator carriers from a second side of the first substrate. The first substrate may include a plurality of inlet holes and a plurality of vent holes extending through the first substrate. One inlet hole may be associated with each of the capsules and may allow liquid to enter the capsule when the device is at least partially submerged in liquid. The vent holes may be spaced from the inlet holes. One of the vent holes may be associated with each capsule and may allow air to exit the capsule as liquid enters the capsule when the device is at least partially submerged in liquid. The device may also have an air channel that extends from each of the capsules and fluidly connects the interior of each of the plurality of capsules and the associated vent hole.

In other embodiments, the device may include an adhesive film located on a second side of the first substrate. The adhesive film may cover the inlet holes and vent holes, and may be removed prior to use of the device to expose the inlet holes and vent holes. The vent holes may be located above a dip line on the first substrate. The first substrate, indicator carriers, and atmospheric isolator(s) may be sealed in a vacuum-pack.

In further embodiments, the atmospheric isolator may include a coating that covers the indicator carriers. The coating may be dissolvable such that it dissolves when the device is immersed in liquid to expose the indicator carriers to the liquid. Additionally or alternatively, the coating may not be dissolvable and the first substrate may have a plurality of apertures extending through the first substrate. The apertures may allow liquid to access each indicator carrier when the device is at least partially submerged in liquid. A dissolvable plug may be located in each of the plurality of apertures. The dissolvable plug may dissolve when submerged in liquid to allow liquid to enter the apertures and contact each of the plurality of indicator carriers.

The device may have a dip line located on the first substrate. The dip line may indicate a depth to which the first substrate should be submerged during use. Each of the indicator carriers may include a reagent secured to a surface of the indicator carrier. The reagent may react with a contaminant of interest in the liquid. The indicator carriers may be test strips.

In accordance with additional embodiments, a method for testing the level of a plurality of contaminants in water includes providing a testing device. The testing device may include a first substrate, a plurality of indicator carriers, and at least one atmospheric isolator covering each of the plurality of indicator carriers. The atmospheric isolator may isolate the plurality of indicator carriers from ambient air prior to at least partial submersion of the substrate in the water. The method may also include (1) at least partially submerging the testing device in water such that water contacts each of the plurality of indicator carriers, (2) removing the testing device from the water, and (3) determining, after a predetermined period of time, if any of the plurality of contaminants within the water exceed a maximum allowable concentration. The method may determine if the contaminant exceeds the maximum allowable concentration based on a color change of each of the plurality of indicator carriers.

The method may further include taking an image of the testing device and each of the indicator carriers. The method may then analyze the image to determine the change of color of each of the indicator carriers and whether a contaminant associated with the given indicator carrier exceeds the maximum allowable concentration. The testing device may include a color calibration reference located on the substrate, and analyzing the image may include performing a color balance on the image.

In some embodiments, the first substrate may be transparent to allow visual inspection of the indicator carriers from a second side of the first substrate. Additionally or alternatively, the atmospheric isolator(s) may include a plurality of capsules, and one of the indicator carriers may be located in each one of the capsules. In such embodiments, the first substrate may include inlet holes and vent holes extending through the first substrate. One of the inlet holes may be associated with each of the capsules and may allow water to enter the capsule when the device is at least partially submerged in water. The vent holes may be spaced from the plurality of inlet holes. One of the vent holes may be associated with each of the capsules and may allow air to exit the capsule as water enters the capsule when the device is at least partially submerged in water. An air channel extending from an interior of each of the capsules may fluidly connect the interior of each of the capsules and the associated vent hole.

The testing device may also include an adhesive film located on a second side of the first substrate. The adhesive film may cover the inlet holes and vent holes. The method may also include removing the adhesive film prior to at least partially submerging the testing device to expose the inlet holes and vent holes. Additionally or alternatively, the testing device may be sealed in a vacuum-pack. In such embodiments, the method may include removing the testing device from the vacuum-pack prior to submerging the testing device.

In accordance with further embodiments, the atmospheric isolator may include a dissolvable coating that covers the indicator carriers. The dissolvable coating may dissolve when the device is immersed in water to expose the indicator carriers to the water. In other embodiments, the first substrate may have apertures extending through the first substrate. The apertures may allow liquid to access each indicator carrier when the device is at least partially submerged in liquid. The device may have a dissolvable plug located in each of the apertures. The dissolvable plug may dissolve when submerged in water to allow water to enter the apertures and contact each of the indicator carriers. The testing device may include a dip line located on the first substrate, and submerging the testing device in water may include submerging the testing device up to the dip line.

### BRIEF DESCRIPTION OF THE DRAWINGS

The foregoing features of embodiments will be more readily understood by reference to the following detailed description, taken with reference to the accompanying drawings, in which:

FIGS. 1A and 1B schematically show an apparatus for water quality assessment, in accordance with some embodiments of the present invention;

FIGS. 2A-2C schematically show an additional embodiment of an apparatus for water quality assessment, in accordance with further embodiments of the present invention;

FIGS. 3A-3D schematically show a further embodiment of an apparatus for water quality assessment, in accordance with further embodiments of the present invention;

FIG. 4 schematically illustrates a container for an apparatus, in accordance with embodiments of the present invention;

FIG. 5A is a flow chart that illustrates a method of use of an apparatus for water quality assessment, in accordance with embodiments of the present invention;

FIG. 5B schematically illustrates an apparatus for water quality assessment disposed in a sample of water to be tested, in accordance with embodiments of the present invention;

FIG. 6 schematically illustrates a system for determining contamination(s) in excess of maximum allowable concentration, in accordance with embodiments of the present invention.

### DETAILED DESCRIPTION OF SPECIFIC EMBODIMENTS

Illustrative embodiments provide an apparatus (or “marker”) that allows for the simultaneous determination of whether any of a large number of potential contaminants (e.g., chemicals; ions) in water are present in a concentration that exceeds the maximum allowable concentration of such contaminants. The design, security and functionality of the model/apparatus do not require a trained operator, or special instruments and conditions in order to use. Any person can easily determine visually, by a color change of indicators, if the elements in the water reached the maximum allowable concentration and/or whether the water is safe to drink. Various embodiments of the present invention may provide for high-speed water quality analysis employing immobilized chemical indicators. They can be used for drinking water, natural water and grey water quality assessment.

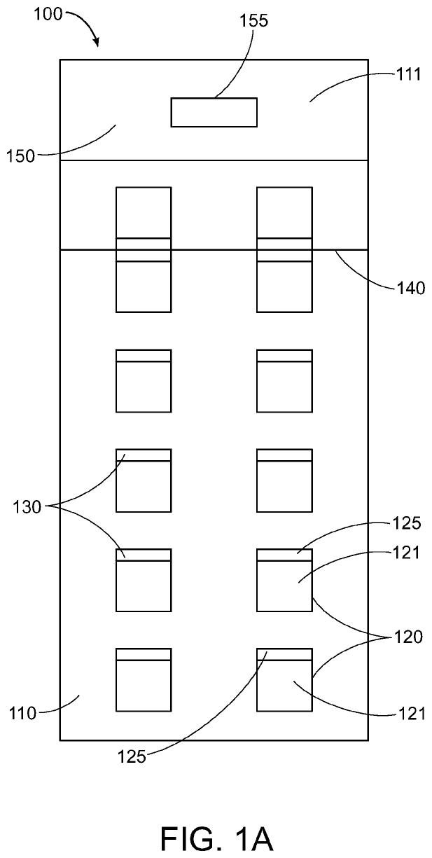

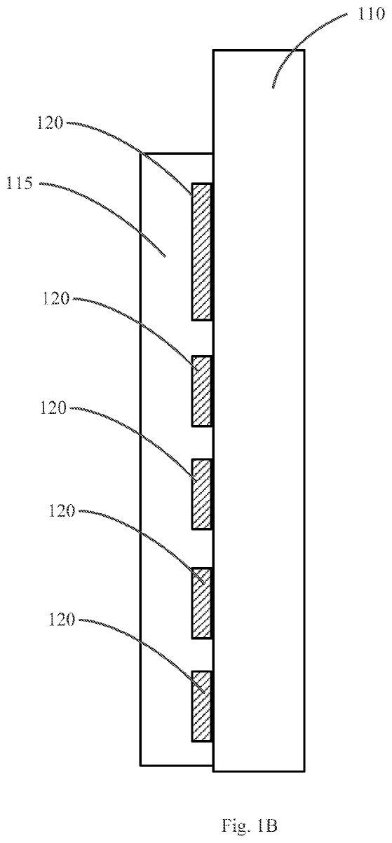

FIG. 1A and FIG. 1B schematically illustrate an embodiment of an apparatus/testing device 100 (which may be referred-to as a “marker,” or as a universal marker for assessment of water quality) for determining the level of a number of contaminants in a liquid (e.g., in a sample of water) and whether the level/concentration of the contaminant(s) exceeds the maximum allowable concentration for that contaminant. Based on this determination, the user may then be able to determine whether or not the liquid/water is safe to consume. For example, if the level of each of the contaminants is below the maximum allowable concentration, the liquid/water should be safe to consume. However, if one or more of the tested contaminants is above the maximum allowable concentration, the liquid/water may not be safe to consume.

As shown in FIGS. 1A and 1B, the marker/testing device 100 includes a substrate 110 that defines the structure of the testing device 100. To allow for better visual inspection of the testing device 100 after use, the substrate 110 may be a transparent film. As discussed in greater detail below, during use, the substrate 110 will be submerged (at least partially) in the liquid to be tested. To that end, the substrate 110 may be semi-rigid or rigid and/or waterproof, so that it may be more easily dipped/submerged into the liquid/water to be analyzed, and so that the testing device 100 will maintain its shape and integrity.

A plurality of indicator carriers 120 (which may also be referred to as “test strips”) are coupled to a front side 111 of the substrate 110. In some embodiments, each indicator carrier 120 may be attached to the substrate 110 at a corresponding attachment point 130. For example, each indicator carrier 120 may be attached to the substrate 110 by a fastener that is not dissolvable and that maintains its integrity when submerged, so that each indicator carrier 120 remains secured to the substrate 110 when the substrate 110, and in particular each indicator carrier 120, is submerged in the water. To that end, the fastener 125 may be a weld, an adhesive, or similar fastener.

To provide a grasping location and help the user hold and manipulate the testing device 100 (e.g., when dipping the device 100 into the liquid and/or removing the device 100 from the liquid), the substrate 110 may include a tab 150, for example, located above each of the indicator carriers 120. The tab 150 may also provide space for inclusion, on the device 100, of a color reference marker 155 (discussed in greater detail below), a logo or other information for a purchaser or user of the device 100.

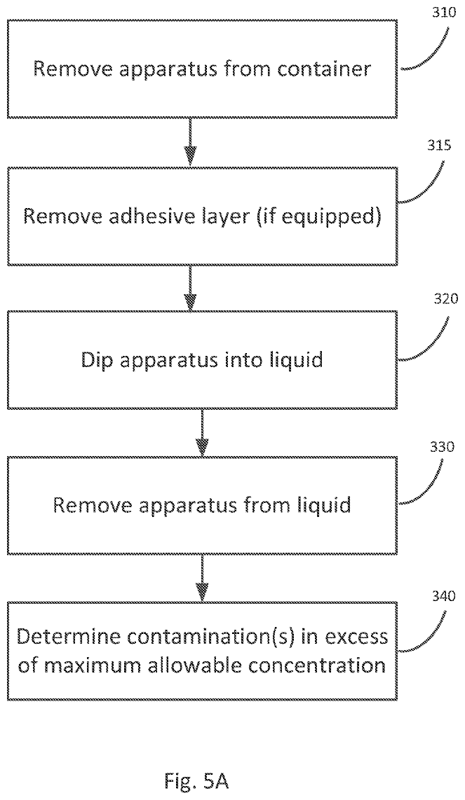

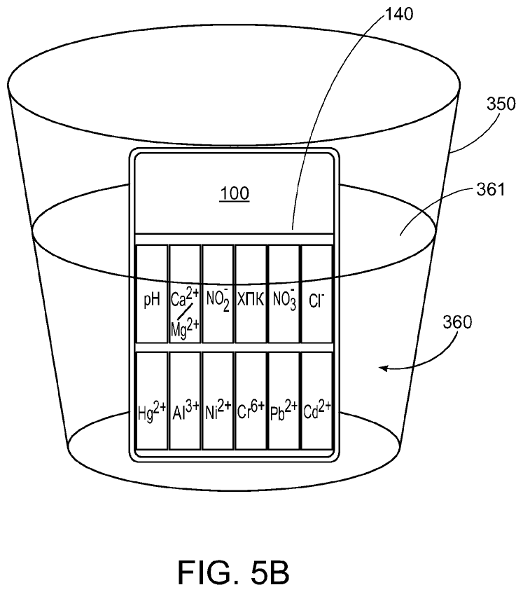

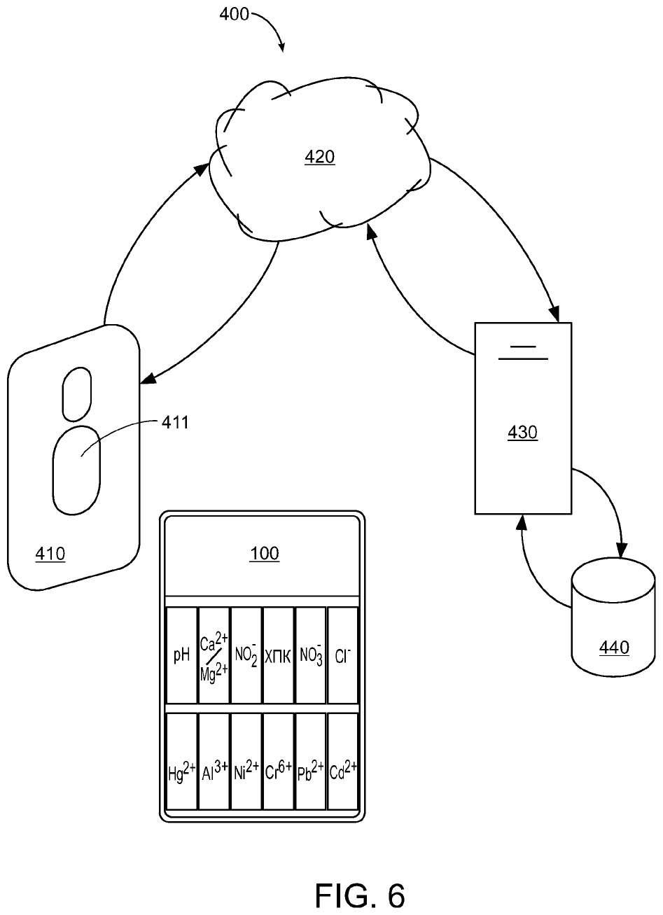

Some embodiments may also include a dip line 140 on the substrate 110. During use, the dip line 140 signals to the user the depth to which the apparatus 100 should preferably be dipped, so that a portion of the substrate 110 below the dip line 140 is submerged (see, e.g., FIG. 5B).

Each indicator carrier 120 includes a reagent 121. The reagent may be affixed to a surface of the indicator carrier 120, or may pervade the indicator carrier 120. The reagent 121 reacts chemically with a potential contaminant of interest in the liquid/water to be tested. For example, each indicator carrier 120 may have a reagent 121 that reacts to a potential contaminant of interest that is different from the potential contaminant of interest on the other indicator carriers 120 so that, collectively, the indicator carriers 120 detect a corresponding number of potential contaminant of interest (e.g., so that one testing device 100 is able to detect numerous potential contaminants of interest).

Some such reagents 121 may also—undesirably—react chemically with ambient air in the vicinity of the water to be tested, and in particular may react to oxygen. Such reactions may undesirably impact the ability of the indicator carrier to detect the contaminants and/or render the reagent unsuitable for use in testing the water, for example by consuming or bonding with some or all of the reagent 121. To prevent such damage and/or reactions with the ambient air, the testing device 100 may include a dissolvable coating 115 (e.g., a polymer coating) on the front face 111 of the substrate that covers the indicator carriers 120, and their respective reagents 121 prior to use of the device. As discussed in greater detail below, during use and when the testing device is submerged in the liquid/water, the coating 115 may dissolve to expose the indicator carriers 120 and reagents 121 to the liquid/water.

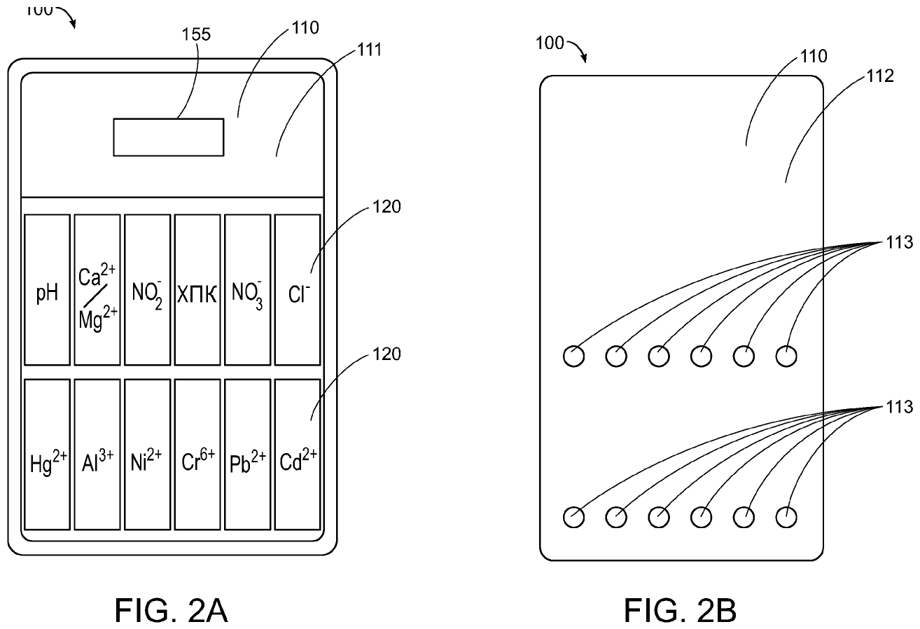

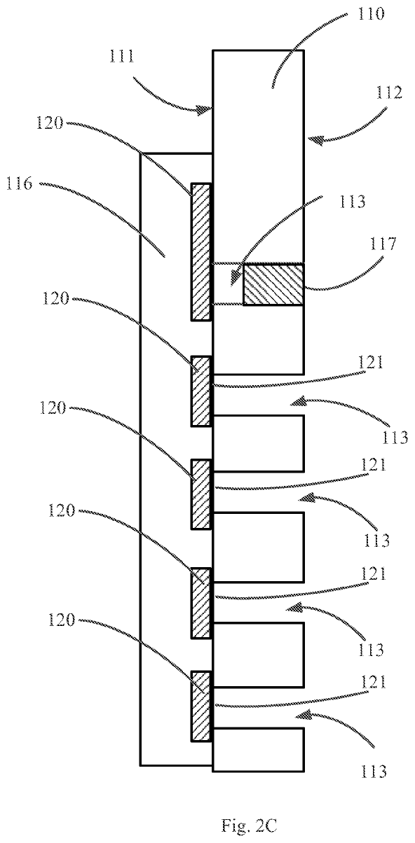

Although the embodiment discussed above has a dissolvable coating 115, other embodiments may utilize a non-dissolvable coating 116 (e.g., a non-dissolvable polymer coating) to protect/seal the indicator carriers 120 and reagents 121 from the ambient air prior to use (see FIGS. 2A-2C). In such embodiments, the testing device 100 may include a number of apertures 113 passing/extending through the substrate. These apertures 113 may be positioned to allow water to access each of the plurality of indicator carriers 120, and in particular to access the respective reagent 121 on each of the plurality of indicator carriers 120. To prevent ambient air from reaching the indicator carriers 120/reagents 121 through the apertures 113, each aperture 113 may be sealed with a dissolvable plug 117. When submerged, the dissolvable plug 117 dissolves, allowing water to pass through the aperture 113 and reach the reagent 121 on the indicator carrier 120. Additionally or alternatively, the testing device 100 may include an adhesive layer/film located on the back side of the substrate 110 and covering the apertures 113. This adhesive layer/film may be removed prior to submerging the testing device to allow the water to enter the apertures 113.

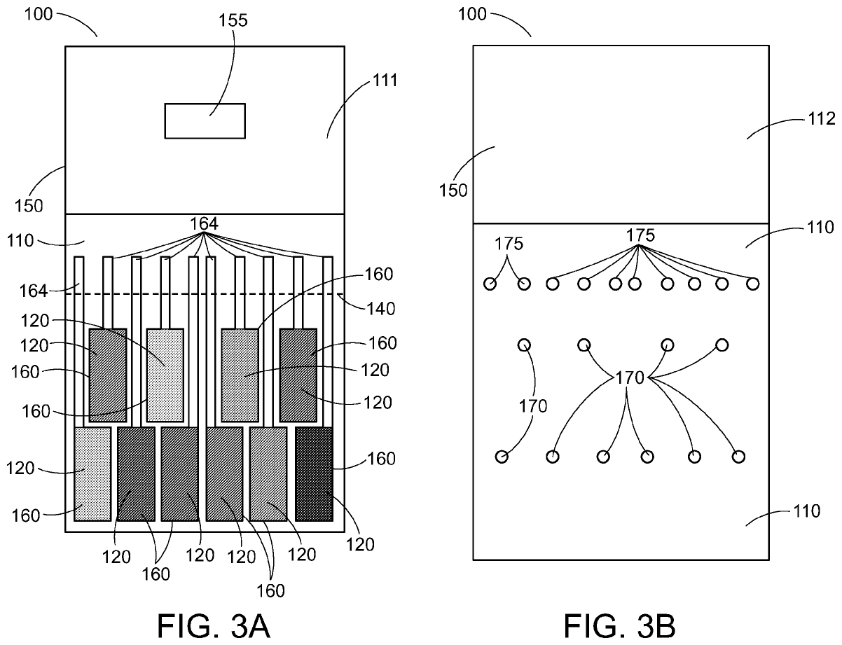

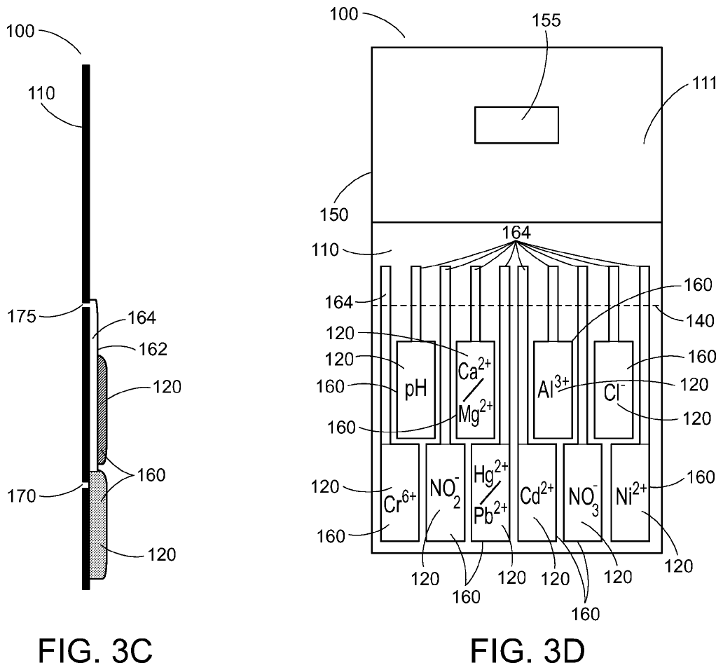

FIGS. 3A to 3D show a further embodiment of a testing device 100 that may be used to test for multiple contaminants in a liquid/water sample with a single device. Like the embodiments described above, the embodiment shown in FIGS. 3A-3D have a substrate 110 that may be dipped/partially submerged in the liquid/water in order to measure the contaminants. The substrate 110 may be transparent to allow the indicator carriers 120 to be viewed and/or imaged (discussed in greater detail below) during analysis. Additionally, the substrate 110 may have a tab 150 for grasping the device 100, a dip line 140 to indicate the appropriate depth to submerge the substrate 110, and a color reference marker 155.

Located on the front/first side 111 of the substrate 110, the testing device 100 may have a number of capsules 160 in which the indicator carriers 120 may be located. For example, the indicator carriers 120 may be attached (e.g., painted on, adhered to, etc.) to an inner wall of the capsule 160 within the interior of the capsule 160. Like the coatings 115/116 discussed above, the capsules 160 help to isolate each of the indicator carriers 120 from the ambient air/environment. To that end, the capsules 160 may be manufactured from water-repellent and chemically inert polymer material. Also, to aid in color analysis after exposing the indicator carriers 120 to the liquid/water, the capsules 160 may be white in color. The white color allows for more convenient and efficient recognition of the indicator carrier 120 color change either by human sight or with an imaging device (discussed in greater detail below).

It should be noted that the capsules 160 may be separate from one another and individually sealed to the front side 111 of the substrate 110. Alternatively, some or all of the capsules 160 may be formed as a single piece. In such embodiments, the capsules 160 may be formed within a second substrate 162 that is secured/sealed to the front side 111 of the main substrate 110. In any event, it should be noted that the interior volumes of each of the capsules 160 should be fluidly disconnected from each other to avoid cross-contamination between the indicator carriers 120 and reagents 121 in each of the capsules 160.

As best shown in FIGS. 3B and 3C, the substrate 110 may have two sets of holes/apertures extending from the back side 112 of the substrate 110 to the front side 111 of the substrate 110. For example, the testing device 100 may have a set of inlet holes 170 and a set of vent holes 170. The inlet holes 170 may be in fluid communication with the interior of the capsules 160 such that the liquid/water may flow through the inlet holes 170 and into the capsules 160 when the testing device 100 is submerged. The vent holes 175 allow air to exit the capsules 160 as they begin to fill with liquid/water. To cover the inlet holes 170 and vent holes 175 prior to use, the device 100 may have an additional layer (e.g., an adhesive layer) that is secured to the back side 112 of the substrate 110 and covers/seals both the inlet and outlet holes 170/175. This additional/adhesive layer may be removed prior to use to allow liquid/water to enter the inlet holes 170 and air to exit the vent holes 175.

In order to prevent liquid/water from accidentally entering the vent holes 175 during use, the vent holes 175 may be spaced from the inlet holes 170 and/or the interior of the capsules 160 and may be located above the dip line 140. To that end, the device 100 may include air channels 164 that extend from the capsules 160 and fluidly connect the interior of the capsule and the vent holes 175. The air channels 164 may be formed in the substrate 110 and/or the second substrate 162 (e.g., along with the capsules 160).

FIG. 3D shows one exemplary arrangement of the testing device 100 and arrangement of the capsules 160 with the indicator carriers 120. This arrangement allows the user to determine the maximum allowable concentration for the contaminants shown in FIG. 3D. For example, the testing device may determine the pH of the water, calcium (Ca²⁺), magnesium (Mg²⁺), aluminum (Al³⁺), chlorine (Cl⁻), chromium (Cr⁶⁺), nitrogen dioxide (NO₂⁻), mercury (HG²⁺), lead (Pb²⁺), cadmium (Cd²⁺), nitrate (NO₃⁻), and nickel (Ni²⁺). These contaminants are the most widespread and dangerous substances polluting water and represent the greatest danger to a living organisms. However, other embodiment, can test for more or less contaminants.

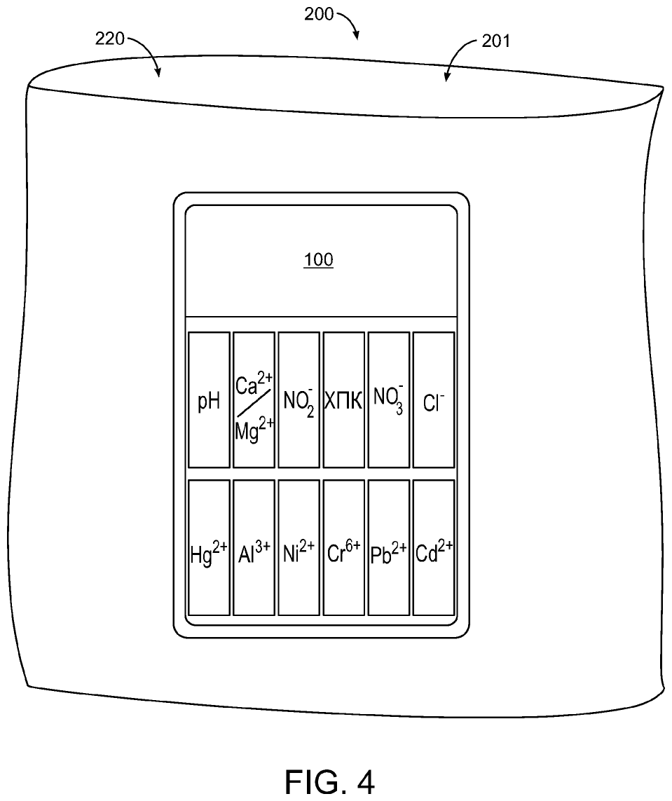

As noted above, ambient air may be harmful to the indicator carriers 120 and reagents 121 prior to use. Therefore, to further protect the testing device 100 prior to use, the testing device 100 may be packaged/sealed within the interior 220 of a hermetic container 200 (see FIG. 4). For example, the hermetic container 200 may be a vacuum container, such that the testing device 100 is kept in a vacuum until the hermetic container 200 is opened. Although a number of hermetic containers may be used, in some embodiments, the hermetic container 200 maybe a sealable bag with an opening 201 through which the testing device 100 may be passed. The opening 201 may then be hermetically sealed. The hermetic container 200 may be a zip-lock bag or other bag with an opening that can be sealed, for example, by an adhesive or heat sealing.

During use of the testing device 100 (see FIG. 5A), the user may first open the hermetic packaging 200 and remove the testing device 100 from the packaging/container 200 (step 310). Once the device is removed from the packaging 200, if the device includes the adhesive layer mentioned above (e.g., the adhesive layer covering the apertures 113 and/or the adhesive layer covering the inlet and vent holes 170/175), the user may remove the adhesive layer (Step 315). As discussed above, removing the adhesive layer (if equipped) will expose the apertures 113 and/or inlet/vent holes 170/175 and allow water to reach the indicator carriers 120 when the device 100 is placed in water. It should be noted that, in order to minimize the exposure to ambient air, it is best to remove the adhesive layer just prior to use.

Once the adhesive layer is removed (if equipped), the user may then dip/submerge the testing device 100 into the liquid/water to be tested so that each of the plurality of carrier indicators 120 is submerged and/or up to the dip line 140. It should be noted that the testing device 100 may be dipped/submerged directly into the source of the water (e.g., into the lake, pond, reservoir, etc.) or a sample of the water may be taken from the source. For example, as shown in FIG. 5B, the user may collect a sample of the water to be tested 360 in a sample container 350. The testing device 100 may then be dipped into the water to be tested 360 up to the dip line so that the dip line is at the surface 361 of the water 360, and the portion of the device 100 below the dip line 140 is submerged.

When the testing device 100 is sufficiently submerged within the water 360, the water 360 will contact the indicator carriers 120. For example, if the device 100 has a dissolvable coating 115 and/or dissolvable plug 117, the coating 115 and/or plug 117 will dissolve and the water 360 will directly contact the indicator carriers 120 and/or flow into the apertures 113 and contact the indicator carriers 120. For those embodiments utilizing capsules 160, the water 360 will enter the interior 162 of each of the capsules 160 via the inlet openings 170 and the air in the capsule 160 will exit the capsule 160 via the vent holes 175 (which may be located above the dip line 140 and/or water surface 161).

The user may leave the apparatus/device 100 within the water 360 for a time that is sufficient to allow each of the reagents 121 to react to its corresponding contaminant (if that contaminant is present in the water 360). In some embodiments, the indicator carriers 120 will change color when in contact with its associated contaminant. The color change (or the extent of the color change) indicates that the contaminant is present in the water 360 at a concentration that meets or exceeds the maximum allowable concentration. In other embodiments (e.g., those with the capsules 160 described above), the indicator carrier 120 may dissolve in the water contained within the capsule 160 to form a solution of reagent and sample water. The time required for the color change or dissolution of the indicator carrier 120 may depend on the application, contaminant of interest, and the indicator carrier 120 and/or reagent 121 used. For example, for embodiments using non-dissolvable indicator carriers 120 that change color, a sufficient time may be one or two minutes. However, for embodiments with capsules 160 and in which the indicator carrier 120 dissolves, 10-20 seconds may be sufficient.

Once a sufficient time has passed, the user may remove the testing device 100 from the water 360 (Step 330). In embodiments using dissolvable indicator carriers 120, the user may also gently shake the device 100 to help the carriers 120 dissolve and may then wait a period of time for the reagent within the capsule 160 to react to its corresponding contaminant if that contaminant is present in the water 360 (e.g., 1-2 minutes). The reagent changes the color of a solution with the sample water when in contact with its associated contaminant. Like with the non-dissolvable carriers 120, the color change of the reagent/water solution indicates that the contaminant is present in the water 360 at a concentration that meets or exceeds the maximum allowable concentration. It should be noted that although times of 1-2 minutes are discussed above, times may be longer or shorter depending, for example, on the contaminant of interest and the reagent 121 used to detect that contaminant.

Once the color change(s) have occurred, the user may then determine for each potential contaminant of interest, whether the contaminant of interest is present in the water 360 at levels or concentrations that meet or exceed the maximum allowable concentration (step 340). For example, in some embodiments of step 320, the user visually observes the indicator carriers 120 to determine which, if any, have changed color. Alternatively, as described in greater detail below, the user may take an image of the testing device 100 and a system 400 (FIG. 6) may determine the color change and/or if the contaminant exceeds the maximum allowable concentration.

As noted above, FIG. 6 schematically illustrates a system 400 for determining contamination(s) in excess of maximum allowable concentrations In operation, the user images the testing device 100 and indicator carriers 120 or capsules 160 after the indicator carriers 120 have been exposed to the water 360. For example, the user may produce such an image using an imaging device 410 (e.g., a camera or smartphone). To that end, in some embodiments, the indicator carriers 120 and/or the interiors of the capsules 160 may be disposed on the substrate 110 such that an image of the indicator carriers 120 and/or the interiors of the capsules 160 may be captured by an imaging device 410 for subsequent analysis of the image.

In some embodiments, the imaging device 410 includes code that, when executed on the imaging device 410, uses a colorimetric algorithm to read the indicator carriers 120 and/or the color of the solution in the capsules 160 and determine whether the indicator carrier 120 and/or solution color indicates that the corresponding contaminant is present in the water 360 at concentrations that meet or exceed the maximum allowable concentration. As part of this analysis, the imaging device 410 may perform a color correction using the color reference 155 on the substrate 110. The imaging device 410 then presents the results of that analysis to the user on a display screen 410. For example, the imaging device 410 may provide the actual concentration levels of each contaminant that is present, a list of those contaminants that are present in the water, and/or provide an indication as to whether the water is suitable for drinking.

In other embodiments, the imaging device 410 may transmit the image, via a network 420, to an analysis server 430. The analysis server 430 includes software that, when executed on the analysis server 430, analyzes each indicator carrier 120 and/or the solution in the capsules 160 to determine whether the indicator carrier 120 and/or solution indicates that the corresponding contaminant is present in the water 360 at concentrations that meet or exceed the maximum allowable concentration. Like the imaging device 410 discussed above, the analysis server 430 may perform a color correction on the image using the color reference 155. The analysis server 430 then communicates its conclusions by sending a report over the network 420 to the imaging device 410. The imaging device 410 then displays the report to the user on a screen 411.

In some embodiments, the imaging device 410 knows its location (e.g., by GPS coordinates), and includes a database 440 that stores records of previous tests of water from that location. The imaging device 410 then compares its conclusion to such records of previous tests and can report to the user the results of such comparison. In other embodiments, the imaging device 410 communicates to the analysis server 430 the location of the imaging device 410. The analysis server 430 then consults a database 440 to find records of previous tests of water from that location. The analysis server 430 then compares its conclusion to such records of previous tests and reports to the user the results of such comparison. It should be noted that the systems 400 described above provide objective, computer analysis and do not require the analysis system to be present at the location of the test.

It should also be noted that various embodiments of the present invention provide numerous benefits over the prior art systems and methods. For example, one benefit and/or technical result provided by the foregoing embodiments is the simultaneous determination of whether each of a plurality of contaminants exceeds a corresponding maximum allowable concentration (MAC) for that contaminant. Additionally, the foregoing embodiments do not require a specialized operator, special instruments, or particular conditions. Instead, any normally-sighted person can determine visually (e.g., by a color change of indicators) that the water includes a concentration of a contaminant in an excess that contaminant's MAC. Moreover, some embodiments do so for contaminants that cannot be identified with visual and organoleptic studies. In preferred embodiments, markers will be packed in to a vacuum pack that provides security and allows a person to carry it safely.

The embodiments of the invention described above are intended to be merely exemplary; numerous variations and modifications will be apparent to those skilled in the art. All such variations and modifications are intended to be within the scope of the present invention as defined in any appended claims. Although various embodiments described herein use water as an example, the apparatuses, methods and systems are not limited to the analysis of water or drinking water.

1. A device for detecting, in a liquid, concentrations of chemicals that exceed a maximum allowable concentrations, the device including:
a first substrate;
a plurality of indicator carriers, each indicator carrier configured to react to a concentration of a chemical that exceeds the maximum allowable concentration for that chemical; and
at least one atmospheric isolator covering each of the plurality of indicator carriers, the at least one atmospheric isolator configured to isolate the plurality of indicator carriers from ambient air prior to immersion of the substrate in the liquid.

2. A device according to claim 1, wherein the at least one atmospheric isolator includes a plurality of capsules, one of the plurality of indicator carriers located in each one of the plurality of capsules.

3. A device according to claim 2, wherein each of the plurality of capsules are sealed to the substrate on a first side of the first substrate.

4. A device according to claim 2, wherein the plurality of capsules are formed in a second substrate, the second substrate sealed to the first substrate on a first side of the first substrate.

5. A device according to claim 2, wherein each of the capsules have an capsule interior, the plurality of indicator carriers located in each of the capsule interiors.

6. A device according to claim 5, wherein each the plurality of indicator carriers is located on an inner wall of a respective one of the plurality of capsules.

7. A device according to claim 5, wherein the first substrate is transparent, thereby allowing visual inspection of the plurality of indicator carriers from a second side of the first substrate.

8. A device according to claim 2, wherein the first substrate includes:
a plurality of inlet holes extending through the first substrate, one of the plurality of inlet holes associated with each of the capsules and configured to allow liquid to enter the capsule when the device is at least partially submerged in liquid; and
a plurality of vent holes spaced from the plurality of inlet holes and extending through the first substrate, one of the plurality of vent holes associated with each of the capsules and configured to allow air to exit the capsule as liquid enters the capsule when the device is at least partially submerged in liquid.

9. A device according to claim 8, further comprising an air channel extending from each of the plurality of capsules and fluidly connecting the interior of each of the plurality of capsules and the associated vent hole.

10. A device according to claim 8, further comprising:
an adhesive film located on a second side of the first substrate and covering the plurality of inlet holes and plurality of vent holes, the adhesive film configured to be removed prior to use of the device to expose the plurality of inlet holes and plurality of vent holes.

11. A device according to claim 8, wherein the plurality of vent holes are located above a dip line located on the first substrate.

12. A device according to claim 1, further including a vacuum-pack, the first substrate, plurality of indicator carriers, and at least one atmospheric isolator sealed in the vacuum-pack.

13. A device according to claim 1, wherein the at least one atmospheric isolator includes a coating covering the plurality of indicator carriers.

14. A device according to claim 13, wherein the coating is dissolvable such that it dissolves when the device is immersed in liquid to expose the indicator carriers to the liquid.

15. A device according to claim 13, wherein the first substrate has a plurality of apertures extending through the first substrate, the plurality of apertures configured to allow liquid to access each indicator carrier when the device is at least partially submerged in liquid.

16. A device according to claim 15, further comprising a dissolvable plug located in each of the plurality of apertures, the dissolvable plug configured to dissolve when submerged in liquid, thereby allowing liquid to enter the plurality of apertures and contact each of the plurality of indicator carriers.

17. A device according to claim 1, further comprising:
a dip line located on the first substrate, the dip line indicating a depth to which the first substrate should be submerged during use.

18. A device according to claim 1, wherein each of the plurality of indicator carriers includes a reagent secured to a surface of the indicator carrier, the reagent configured to react with a contaminant of interest in the liquid.

19. A device according to claim 1, wherein the indicator carriers are test strips.

20. A method for testing the level of a plurality of contaminants in water comprising:
providing a testing device, the testing device including:
a first substrate,
a plurality of indicator carriers, and
at least one atmospheric isolator covering each of the plurality of indicator carriers, the at least one atmospheric isolator configured to isolate the plurality of indicator carriers from ambient air prior to at least partial submersion of the substrate in the water;
at least partially submerging the testing device in water such that water contacts each of the plurality of indicator carriers;
removing the testing device from the water; and
determining, after a predetermined period of time, if any of the plurality of contaminants within the water exceed a maximum allowable concentration based on a color change of each of the plurality of indicator carriers.

21-35. (canceled)
